(19) United States
(12) Patent Application Publication
(10) Pub. No.: US 2004/0073021 A1
(43) Pub. Date: Apr. 15, 2004

(54) Cloning and sequencing of the porcine interleukin-12 receptor beta-1 and beta-2 chains

(76) Inventors: Federico A. Zuckermann, Doverbrook, CT (US); William Schnitzlein, West Healy, IL (US)

Correspondence Address: Barnes & Thornburg, PO Box 2786, Chicago, IL, 60690-2786, US

(21) Appl. No.: 10/398,666

(22) PCT Filed: Oct. 11, 2001
(86) PCT No.: PCT/US01/32088

(51) Int. Cl. C12Q 1/68; A61K 49/00; A01K 67/027; C07H 21/04; C07K 14/715

(52) U.S. Cl. 536/23.5; 435/6; 435/69.1; 435/320.1; 435/325; 530/350; 424/9.2; 800/17

(57) ABSTRACT

The genes encoding for the porcine beta-1 and beta-2 chains of the porcine interleukin-12 receptor have been cloned. The complete sequence of complementary DNA of both chains has been determined. Variants have been found associated with superior cellular immunity. Applications of the novel molecules are discussed.

### BACKGROUND OF THE INVENTION

The genetics of the porcine IL-12 receptor are determined.

The genes encoding the porcine beta-1 and beta-2 chains of the porcine interleukin-12 receptor were cloned. The complete sequence of the complementary DNA of both chains was determined. Variants (polymorphisms) were identified that are correlated with immune function.

Interleukin-12 (IL-12) is a heterodimeric cytokine consisting of two subunits having molecular weights of 35 and 40 kDa. Both subunits are highly conserved across species, with the predicted amino acid sequences of the two porcine IL-12 subunits being approximately 85% homologous with their human counterparts (Foss and Murtaugh, 1997). IL-12 is the single major factor that is required for the efficient differentiation of naive T cells into memory/effector T-cells capable of producing IFN-gamma. Also known as an NK cell stimulatory factor, IL-12 is a cytokine with clear pro-inflammatory functions. IL-12 stimulates the proliferation of activated T and NK cells, and also stimulates the lytic activity of both cytotoxic T lymphocytes and NK cells (Romani, et al., 1997; Trinchieri and Scott, 1994; Trinchieri, 1997). The biological activity of IL-12 is mediated via binding to specific cell surface receptors.

The human IL-12 receptor consists of a disulfide-linked oligomer composed of two beta-type cytokine receptor subunits termed IL-12 receptor beta-1 and IL-12 receptor beta-2 and having an estimated molecular weight of 130 kDa each. Together these two subunits give rise to the high affinity IL-12 receptor, whereas each independently expressed chain exhibits low affinity for IL-12 (Presky et al., 1996, 1998). The expression of both IL-12 receptor beta-1 and IL-12 receptor beta-2 subunits is upregulated following activation in CD4⁺, CD8⁺ lymphocytes as well as in NK cells (Szabo et al., 1997; Wu et al., 1997). However, expression of the two IL-12 receptor subunits appears to be differentially regulated, at least at the transcriptional level. Although the production of mRNA for both chains is induced during the activation of T cells, transcription of the IL-12 receptor beta-2 gene appears to be more tightly regulated, in that only this subunit, but not the beta-1 subunit is inhibited by IL-10 and TGF-beta (Wu et al., 1997). The biological activity of IL-12 is dependent on the cell surface expression of the receptors for this cytokine. Allelic variants of this beta-1 receptor have been shown to affect the ability of humans to develop a protective immunity against intracellular pathogens such as Salmonella and Mycobacteria.

Interleukin-12 pays a major role in determining the nature of the immunity that develops in response to infection or vaccination against an infectious agent. This effect is mediated through the binding of this cytokine to its cell surface receptor on lymphoid cells.

If genetic variants of IL-12 receptors exist in other species, selective breeding could improve animal herd health. Because IL-12 plays a key role in regulating the development of cell-mediated immunity, it is possible that within the swine population there are animals that possess IL-12 receptor alleles that have a superior ability to respond to the differentiation signals provided by IL-12. These animals may consequently be genetically predisposed to develop a strong cellular immune response upon infection or vaccination against a pathogen. Superior cellular immune response is likely to provide a higher level of protective immunity against infectious diseases in where this type of immunity plays a role in protection from disease. What is needed is to develop a genetic marker that will identify animals such as swine capable of developing a strong cellular immune response to a given pathogen.

### SUMMARY OF THE INVENTION

The present invention relates genetic markers that can identify swine with superior cellular immunity. The markers are allelic variants of the IL-12 receptor gene, which consists of 2 subunits.

An aspect of the invention is a porcine IL-12 receptor encoded by the cDNA molecules disclosed herein. A cDNA molecule encoding the porcine beta-1 chain of the porcine interleukin-12 receptor has a nucleotide sequence as shown in FIGS. 1 and 2. A molecule with an amino acid sequence deduced from the cDNA sequence of the beta-1 chain is also shown in FIGS. 1 and 2. A variant of a cDNA molecule encoding the porcine beta-1 chain of the porcine interleukin-12 receptor which lacks 37 nucleotides from positions 628-664 and results in a premature termination of the IL-12R beta portion as shown in FIGS. 3 and 4. A molecule with an amino acid sequence deduced from the cDNA sequence is also shown in FIGS. 5 and 6. A cDNA molecule encoding the porcine beta-2 chain of the porcine interleukin-12 receptor has a nucleotide sequence as shown in FIGS. 5 and 6. A molecule with an amino acid sequence deduced from the cDNA sequence is also shown in FIGS. 5 and 6. This molecule is present in pigs found to have a high IFN-g response or cell mediated immunity to an infectious agent.

An aspect of the invention is a molecule with an amino acid sequence having an allelic variant at position 1254 that results in a serine at amino acid position #342 as shown in FIG. 5. The variant results from a nucleotide change at position 1254 from adenine to cytosine. The variant is present in pigs found to have a high IFN-gamma response or cell-mediated immunity to an infectious agent PRRSV (Porcine Reproductive and Respiratory Syndrome Virus) in a pig.

The invention also relates a method of identifying pigs capable of developing a strong cellular immune response to a given pathogen. The method includes the steps of:

(a) specifying a given pathogen; and

(b) selecting pigs with allelic variants of an IL-12 receptor that confer on the pigs a superior ability compared to pigs without the variants, to develop a strong cellular response upon infection or vaccination against the pathogen.

An aspect of the invention is a method for improving the cellular immune response of pig herds. The method includes the following steps:

(a) selecting pigs with allelic variants of an IL-12 receptor that confer on the pigs a superior ability compared to pigs without the variants, to develop a strong cellular response upon infection or vaccination against a pathogen; and

(b) breeding the selected pigs according to methods known to those of skill in the art of pig breeding to produce pigs with improved cellular immunity.

### BRIEF DESCRIPTION OF THE DRAWINGS

FIG. 1 shows the cDNA sequence (SEQ ID NO: 7) for the pig IL-12R beta-1 chain. The nucleotide sequence is on the bottom with the deduced amino acid sequence (SEQ ID NO: 8) on top. A variant of the IL-12 receptor beta-1 chain that results in a premature termination of the coding sequence and a truncated protein has a deletion (underlined). The resulting sequence follows in order the full sequence of the beta-1 chain. A portion of a gene is deleted in the variant resulting in a stop codon downstream. This results in premature termination of protein translation.

FIG. 2 is the nucleotide sequence of FIG. 1 (SEQ ID NO: 7) in a more compact form without the amino acid sequence on top.

FIG. 3 is a variant that lacks 37 nucleotides from positions 628-664 in the pig IL-12R beta-1 cDNA (SEQ ID NOS: 9 and 10). The deletion results in premature termination of the IL-12R beta-1 protein. The nucleotide sequence of the variant extends from positions 58-2318 in FIG. 1 in the pig IL-12R beta-1 cDNA (position 1 in FIG. 3 is position 58 in FIG. 1).

FIG. 4 is FIG. 3 (SEQ ID NO: 9) without the amino acids corresponding to the nucleotide code.

FIG. 5 shows the amino acid and corresponding nucleotide sequence for pig IL-12R beta-2 cDNA (SEQ ID NOS: 11 and 12). A variation on the beta-2 chain changes the amino acid sequence from a tyrosine to a serine. There is a mutation (polymorphism) from A→C (underlined) at position #1254 that changes the amino acid encoded from tyrosine to serine. The change results in a non-conservative replacement of tyrosine by serine which causes an increased response to infectious agents.

FIG. 6 is FIG. 5 (SEQ ID NO: 11) showing only the nucleotide sequence.

FIG. 7 shows the intensity of the cellular immune response of swine following immunization with a PRRSV MLV vaccine. Twenty-four full-sibling pigs (shown in Table 1) were immunized at 9 and 12 weeks of age with a PRRSV MLV vaccine. Peripheral blood mononuclear cells were isolated from them at 2 weeks after the second immunization and stimulated with PRRSV virus for 20 hours. The frequency of IFN-gamma secreting cells was determined with an IFN-gamma ELISPOT assay as described in the Materials and Methods section herein (Zuckermann et al., 1988). The results obtained were separated into two groups according to the genotype of the responder pig into IL-12R beta-2^{c/c} (8 pigs), IL-12R beta-2^{a/c} (9 pigs), IL-12R beta-2^{a/a} (7 pigs). The data for these four weeks was analyzed by ANOVA (Analysis of Variance).

### DESCRIPTION OF THE INVENTION

The present invention relates methods and compositions to identify pigs with superior cell-mediated immunity based on their IL-12R genotype. The receptor and its coding sequences were identified. cDNA sequences and deduced amino acid sequences of the 2 subunits forming porcine IL-12 receptor are disclosed. These cloned porcine genes and predicted immune response of pigs due to IL-12 receptor genotype differences, have not been previously reported.

Information obtained on the full sequence of both chains of the porcine IL-12 receptor, is useful to identify allelic variants of these genes that differ in their function. Because IL-12 binds to its receptor on the surface of T cells, structural (sequence) variations on the gene may affect the functionality of the receptor. The invention relates to effects of allelic variations on the functionality of the IL-12 receptor which are likely to have an impact on the ability of a pig to develop a cellular immune response to an infectious agent. As discussed in the Background, IL-12 is known to control the development of cell-mediated immunity.

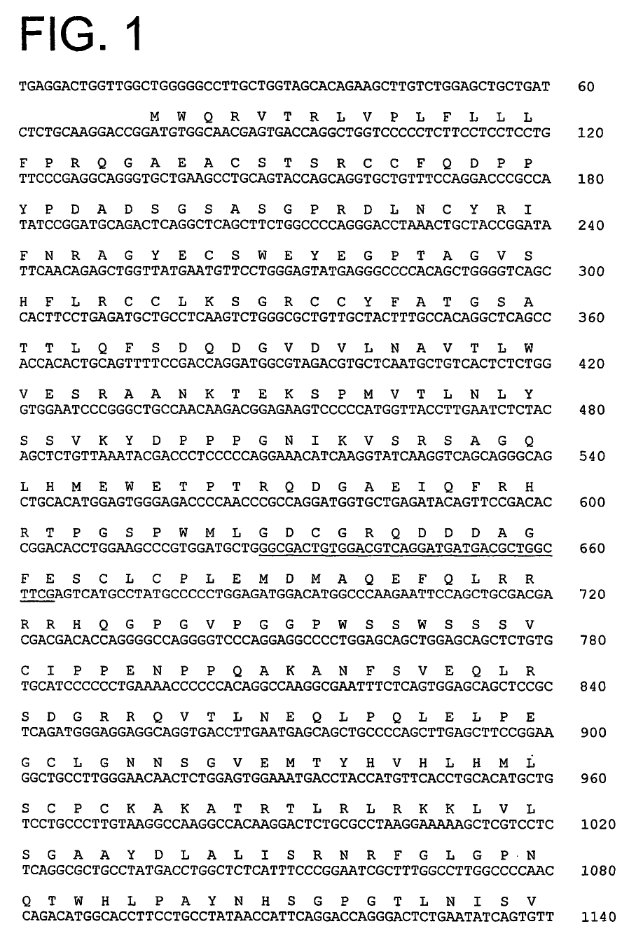

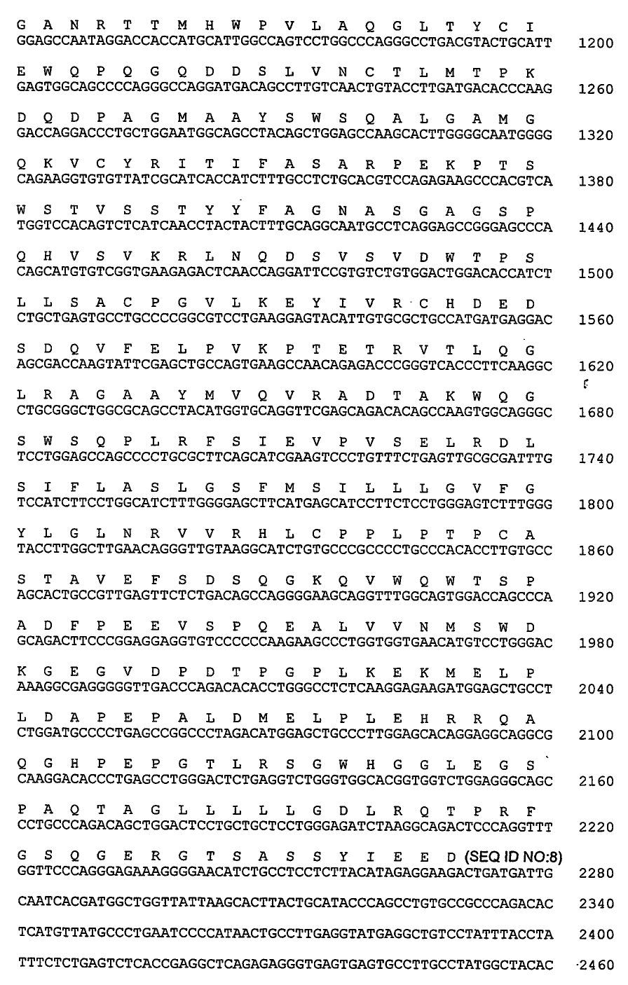

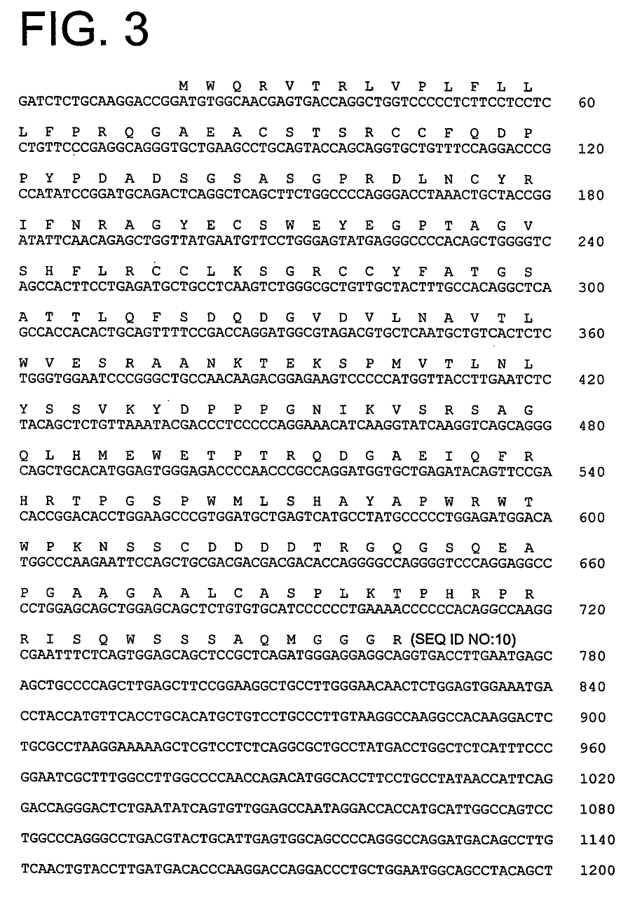

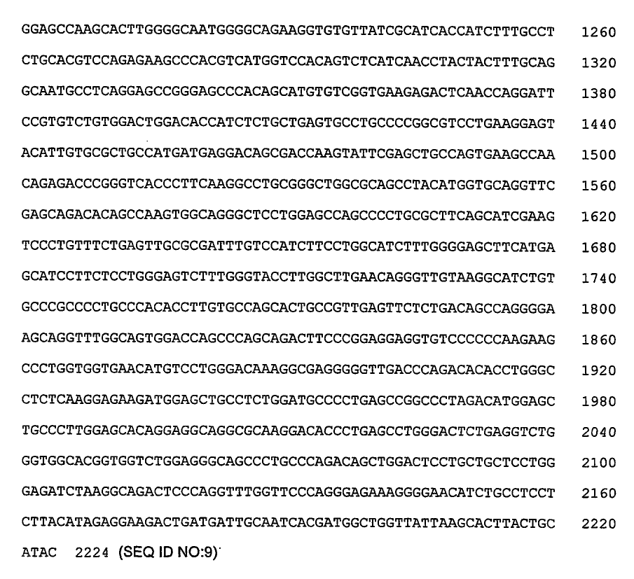

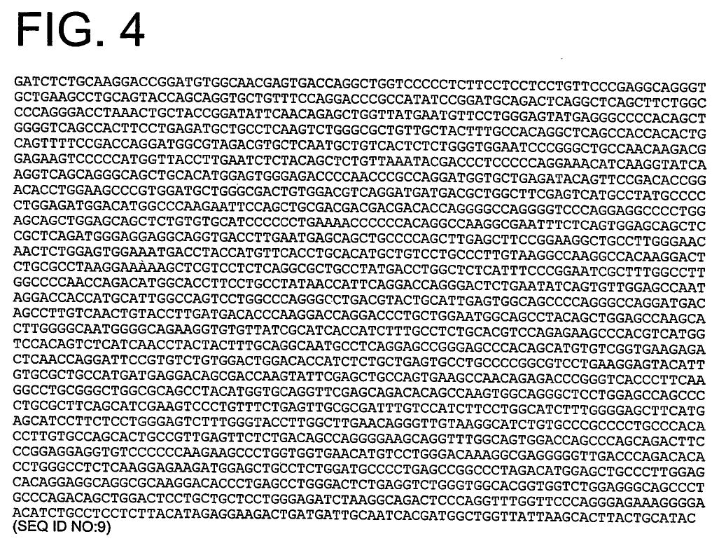

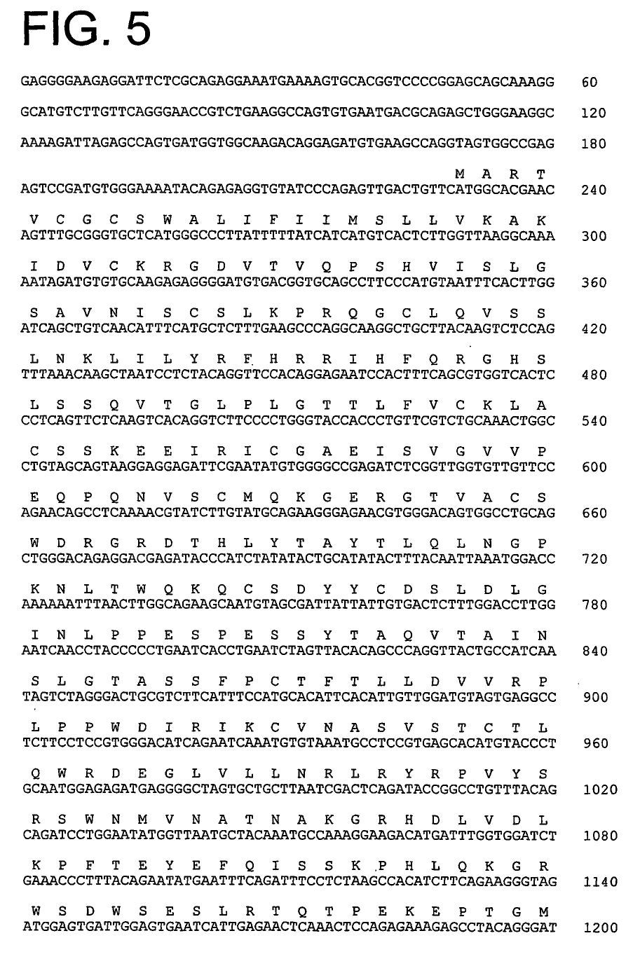

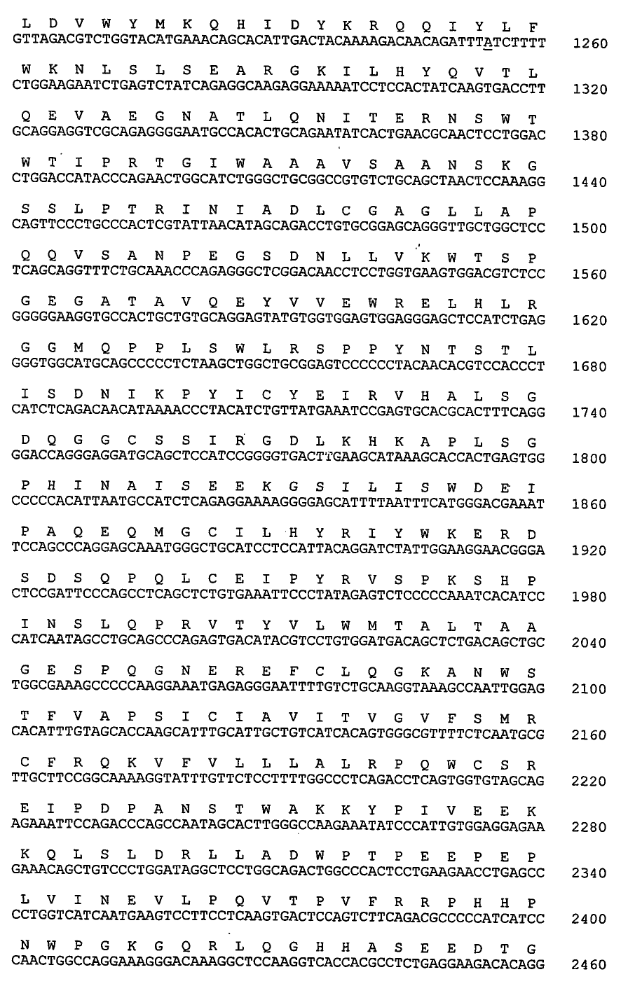

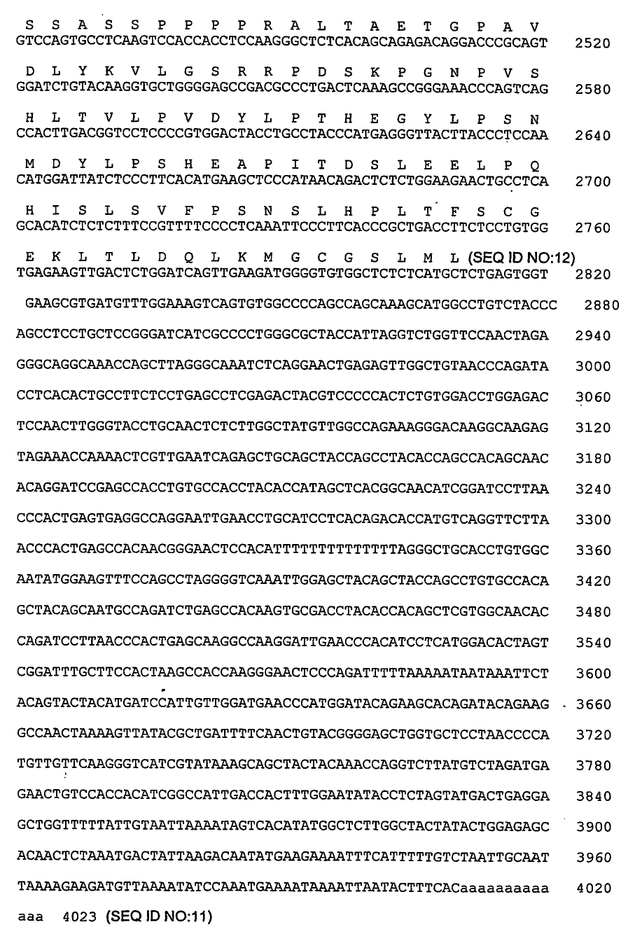

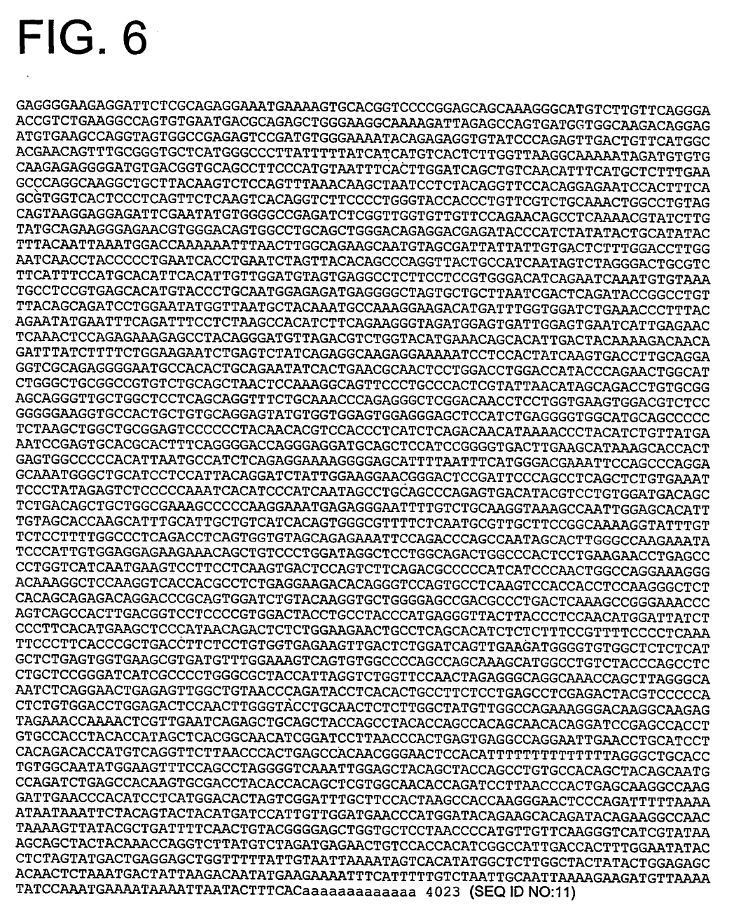

The cloning and sequencing of the porcine IL-12 receptor is a first step in the identification of animals with superior cellular immunity. Having obtained the sequence of the genes encoding for the two chains of the receptor (FIGS. 1-4, showing the beta-1 chain and FIGS. 5 and 6 showing the beta-2), allelic variants of these genes were identified that allow for a higher level of a cellular immune response to microorganisms as compared to pigs without the variants.

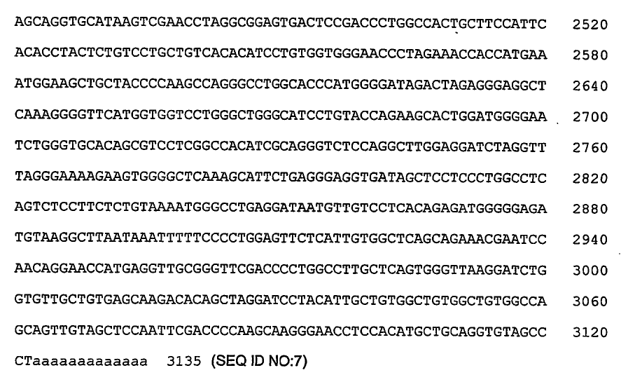

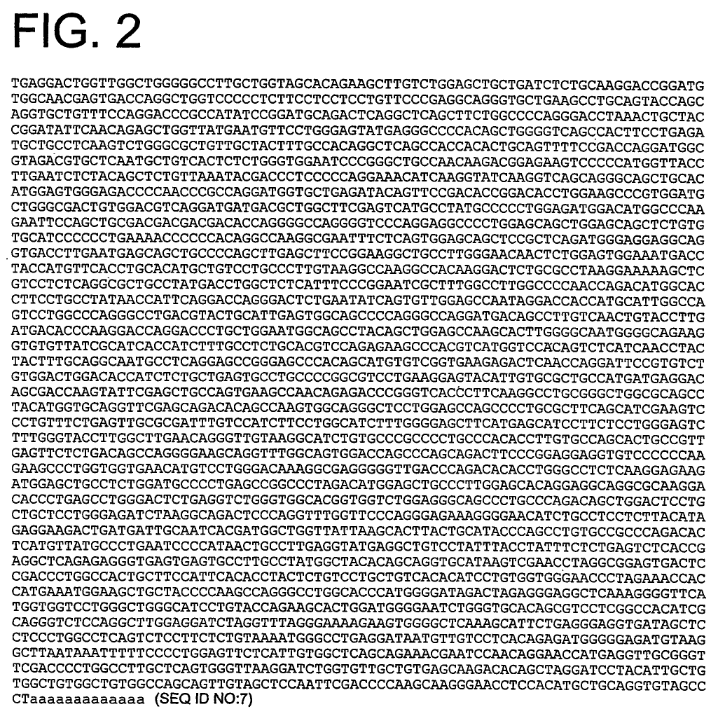

The IL-12 receptor consists of two chains (subunits), the IL-12R beta-1 and IL-12 beta-2 chains. Each chain was individually cloned and sequenced. FIGS. 1-4 show the DNA sequences of the IL12R beta-1 chain. A variant of the IL-12 receptor beta-1 chain was discovered which results in premature termination of the coding sequence. In this variant, the resulting protein translation terminates prematurely (see FIGS. 1 and 2). The variant of the IL-12R beta-1 chain lacks 37 nucleotides from positions 628 through 664. This deletion results in a premature termination of the IL-12R beta-1 protein.

To determine the IL-12R beta-2 cDNA sequence, four groups of pigs were obtained from the Pig Improvement Company (PIC). Each group of pigs was sequenced to determine the IL-12R beta-2 cDNA. These sequences were compared and the following polymorphisms were determined: Position 245 (G to T changes: Glycine to Cysteine. Non-conservative replacement. Located in leader sequence); Position 556 (T to G changes: Aspartic acid to Glutamic acid. Conservative replacement); Position 890 (A to G changes: Isoleucing to Valine. Conservative Replacement); Position 1254 (A to C changes: Tyrosine to Serine. Non-conservative replacement); Position 2233 (C to T changes: Aspartic acid to Aspartic acid. No change in identity); and Position 2440 (C to T changes: Histidine to histidine. No change in identity).

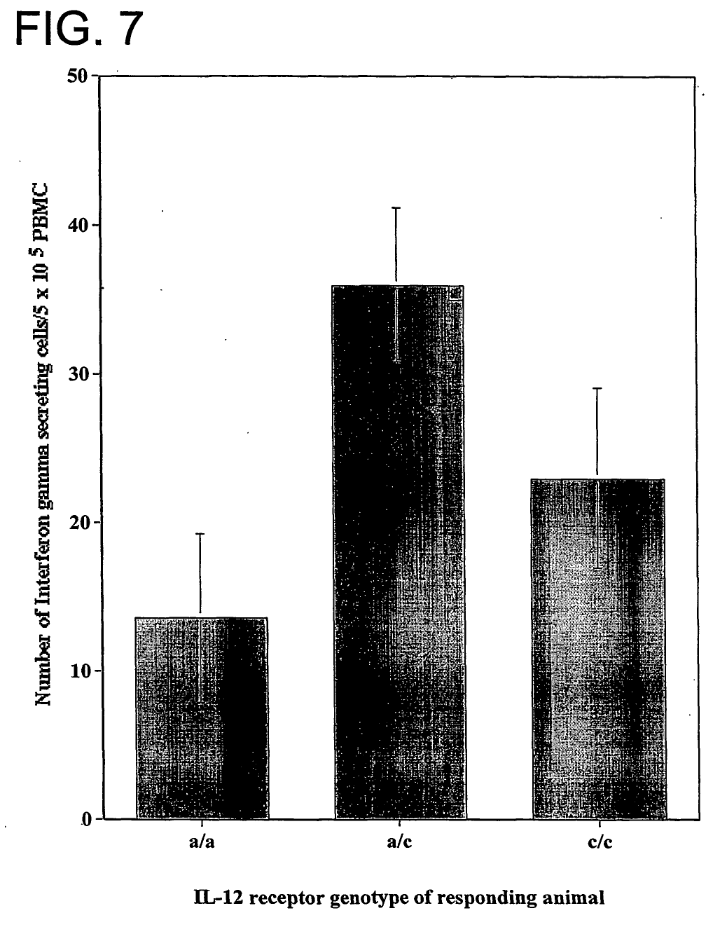

Allelic variation of the IL-12 receptor beta-2 (IL-12RBeta-2) chain of this molecule has an impact on the ability of a pig to develop a cellular immune response to an infectious agent. Of all the variants discovered, a polymorphism at position 1254 (a nucleotide change from A to C) within the porcine IL-12 beta-2 subunit gene) (FIGS. 5 and 6), which results in either a serine or a tyrosine at amino acid position #342 of the intact swine IL-12R beta-2 chain was of particular interest. This change has an impact on the intensity of the cell-mediated immune response to an infectious agent. Only the former amino acid has been detected at the corresponding site in IL-12R beta-2 of either human, murine or bovine origin. The IL-12 beta-2 allele expressing a serine at nucleotide position 1254 in pigs is associated with a greater host interferon-gamma response to immunization with porcine reproductive and respiratory virus (FIG. 7). The expression of this allele is likely associated with a higher interferon gamma response to other microorganisms of swine in general.

Initially, the virus-specific immunity elicited in a group of pigs in response to vaccination with a modified live virus (MLV) vaccine against porcine reproductive and respiratory syndrome virus (PRRSV) was evaluated. The MLV is a non-virulent attenuated vaccine manufactured by Boehringer Ihgelheim Vet Medica. The PRRSV is a stock arterivirus (strain VR2332 from the American Type Culture Collection). One pig from this injection experiment (designated No. 785g) was unique. In this animal the frequency of PRRSV-specific IFN secreting cells in the peripheral blood elicited in response to immunization with this virus was more than double that of any of the other nine pigs in the group. Thus, this pig appeared to be a high responder to PRRSV antigens. To extend this observation and determine the genetic basis for the high responder phenotype the mother and father of pig 785g were bred and produced 24 full siblings to this original animal. The genotype of these pigs for the IL-12R beta-2 chain at positions 1254 is shown in Table 1.

TABLE 1
IL-12R Beta-2 Genotype of Full-Siblings of Pig 785 g.

| | |
|---|---|
| Litter 1 | Litter 2 |

| Pig. No. | Genotype* | Pig. No. | Genotype |
|---|---|---|---|
| 1 | a/c | 211 | a/a |
| 2 | c/c | 212 | a/a |
| 3 | a/a | 213 | c/c |
| 4 | c/c | 214 | a/c |
| 5 | c/c | 216 | a/c |
| 6 | a/a | 217 | a/c |
| 7 | c/c | 218 | a/c |
| 8 | a/c | 219 | c/c |
| 9 | c/c | 222 | a/a |
| 10 | c/c | 221 | a/c |
| 12 | a/c | 222 | a/c |
| 13 | a/a | 223 | a/a |

*The genotype indicated the allele present at the IL-12R beta-2 loci. a = tyrosine and c = serine in amino acid #342 of the porcine IL-12R beta-2 chain. A change in nucleotide position 1254 from A to C results in a non-conservative replacement of amino acid 342 from tyrosine to serine.

Using the pigs described in Table 1 animals expressing a “c” allele for the IL-12R beta-2 were more likely to have the ability to develop a high virus-specific IFN-gamma response following an immunization with a PRRSV modified live virus vaccine than those not expressing this allele. As shown in FIG. 7, the virus-specific IFN-gamma response of pigs with the IL-12R beta-2^{a/c} genotype was significantly higher (p<0.01) than pigs with the IL-12R beta-2^{a/a} genotype at 6 weeks following vaccination. Although the IFN-gamma response of pigs with the IL-12R beta-2^{c/c} genotype was not significantly higher than that of pigs with the IL-12R beta-2^{a/a} genotype, it followed the same high response trend seen with the IL-12R beta-2^{a/c} pigs.

Materials and Methods

Method for Differentiation of “A” and “C” Homozygotes and Heterozygotes to Nucleotide Position 1254 of the Sequenced Porcine IL-12R Beta-2 cDNA.

To identify the polymorphism associated with nucleotide position 1254 of the porcine IL-12R Beta 2 cDNA, primers were engineered to create either one or two novel EcoR I sites in the amplicon generated during amplification of a portion of this template. The forward PCR primer (SEQ ID NO: 1) (GACTACACAAGACAACAGAATT) is nearly identical to the nucleotide stretch (SEQ ID NO: 2) (GACTACAAAAGACAACAGATTT) immediately upstream of the polymorphic site. Use of this primer results in the third upstream thymidine being converted to an adenosine moiety. Thus, when a cytosine is present at the polymorphic site, an EcoR I recognition sequence (GAATTC) is generated. When an adenosine is present, the EcoR I recognition sequence is not made. The reverse PCR primer (SEQ ID NO: 3) (TATTATGCAGTGTGGAATTCCC; complement—(SEQ ID NO: 4) GGGAATTCCACACTGCATAATA) is nearly complementary to a nucleotide stretch (SEQ ID NO: 5) (GGGAATGCCACACTGCAGAATA) downstream of the polymorphic site. Use of this primer results in the conversion of the guanosine at position seven to a thymidine moiety and the creation of an EcoR I recognition sequence.

Differentiation of the A/C polymorphism is accomplished in the following manner. First, RNA is isolated from swine leukocytes previously activated by exposure to a mitogen such as phytohemmaglutinin. It should be noted that since IL-12R Beta 2 transcripts are not present in detectable levels in quiescent cells, their production needs to be stimulated. Then, the RNA is reverse transcribed by using random hexamers as primers and a portion of the resultant IL-12R Beta 2 cDNAs are amplified by a “proof-reading” polymerase. Amplicons generated by the two above mentioned primers are 127 bp in length and contain either one or two acquired EcoR I recognition sites depending on whether an adenosine or cytosine moiety is present at the polymorphic site, respectively. Digestion of the products with EcoR I yields either a 110 (adenosine) or 90 (cytosine) bp product which can easily be identified based on their relative mobilities in polyacrylamide gels.

Method for Pig IFN-Gamma Elispot Determination of Cell-Mediated Immunity

The magnitude of the cellular immune response to immunization with the PRRS virus vaccine was quantified by utilizing an IFN-ELISPOT assay. Briefly, 96-well Immulon II™ plates (Dynatech Inc.) were coated with 50 ml per well of a 24 μg/ml solution of mAb P2G10 in 0.1 M carbonate buffer, pH 9.6. After an overnight incubation at 4° C., each well was washed three times with sterile PBS and then incubated with 50 μl of RPMI supplemented with 5% fetal porcine serum for two hours at 37° C. in a 5% COPeripheral blood mononuclear cells (PBMC) from vaccinated or control pigs were plated at 5×10⁵ viable cells per well. In all samples, PBMC were >98% viable as confirmed by vital dye exclusion. The in vitro recall response to PRRSV was stimulated by the addition of antigen in the form of live virus. PBMC were exposed to viral antigen at 37° C. in a 5% CO by washing the wells six times with phosphate buffered saline (PBS) supplemented with 0.05% Tween-20. Fifty μl of 0.25 μg/ml biotin-labeled mAb P2C11 in 0.05% PBS-Tween was then added to each well and the plates were incubated for 1 hour at 37° C. After washing, 50 μl of 0.31 μg/ml of streptavidin-horseradish peroxidase (SA-HRP; Zymed; San Francisco, Calif.) was added to each well and the plates were incubated for an additional hour at 37° C. Excess SA-HRP was removed by washing the wells 3 times and then 50 μl of TMB membrane peroxidase substrate (Kirkegaard & Perry Laboratories, Gaithersburg, Md.) was added to each well. Hydrolysis of this compound results in the development of blue spots whose size and intensity are directly proportional to the amount of bound IFN-. The frequency of virus-specific IFN-SC was determined by enumerating the blue spots.

Method for Selecting and Breeding Pigs to Improve Immunity

In order to select pigs for breeding which express the improved immunity, the pigs will first be screened through the method for differentiation of A and C homozygotes and heterozygotes at nucleotide position 1254 of the sequenced porcine IL-12R Beta-2 cDNA as previously stated in the Materials and Method section. After that screening process has occurred, pigs will be selected which contain the C marker. After selection these pigs will be bred by conventional methods known to those of skill in the art of pig breeding to increase the frequency of the “C” polymorphism in the herd.

Method of Identifying Polymorphisms in the Porcine IL-12R Subunits

To identify polymorphisms within the two porcine IL-12R subunit genes, the intact open reading frames encoding these entities were determined by sequencing overlapping RT-PCR products derived from the respective transcripts. Initially, the source of RNAs was from activated T cells obtained from three pigs selected at random from each of the four above mentioned groups of swine provided by POC. For comparison, similar determinations were made for pigs derived from GENETIPORC breeding stock (University of Illinois Veterinary Medicine Swine research Farm). These animals had been participants in an unrelated experiment evaluating immunity elicited in response to vaccination with a PRRS modified live virus (MLV). Within this group of ten, pig 758g was unique in that the frequency of its PRRS virus-specific IFN-gamma secreting cells was more than double that of any of the other nine pigs. Moreover, the novelty of pig 758g's response was even more apparent in view of the fact that immunization with a PRRS MLV vaccine has been shown to elicit a weak cell-mediated immune response. In this regard, the frequency of IFN-gamma secreting cells usually ranges between 50-150 per million mononuclear cells. Thus, based on these values, pig 758g was considered to be a high responder whereas pig 834b for example was considered to be a low responder to recall PRRS virus antigens. Due to the divergence in their immune response phenotypes, the predicted primary structures of the IL-12 receptors were determined.

Method of Discovering Receptors

Initially, pairs of degenerate primers, representative of conserved sequences in cDNA encoding the beta-1 and beta-2 subunits of the mouse and human IL-12 were used to amplify internal portions of the respective porcine cDNAs by RT-PCR. Subsequently, the remainder of both cDNAs was obtained by 5′ and 3′ RACE combined with “primer walking.” Both entities have been completely sequenced in both directions. The coding portion of the IL-12R beta-1 cDNA is 2196 nucleotide, while that for IL-12R beta-2 is 2583 nucleotides.

Both chains of the IL-12R are members of the class I cytokine receptor family which includes gp130, granulocyte colony stimulating factor receptor, and leukemia inhibitory factor receptor. All members of this family share several structural characteristics. Accordingly, both of the porcine IL-12R chains have these characteristics which include a Cys-Cys pair (C/CxW) motif in the amino terminal part of their extracellular domain and a (SEQ ID NO: 6) Trp-Ser-x-Trp-Ser (WS x WS) motif in the C-terminal portion. Presumably, these sequences contribute to ligand interaction and protein architecture. Moreover, the intracellular portion of the porcine IL-12R beta-2 chain contains three tyrosine conserved in the human and mouse counterparts. In these two species, these three tyrosines become phosphorylated upon triggering of the IL-12R and induce activation of quiescent cytoplasmic transcriptional factors. In addition, the intracellular domain of both porcine IL-12R chains has two regions of amino acids, termed box 1 and box 2, which are required fro signal transduction. Box 1 is comprised of a Pro-X-Pro sequence preceded by a cluster of hydrophobic amino acids. The second motif, box 2, has a distinct sequence that is not conserved in all members of the family. The presence of all these structural features demonstrates unequivocally that the identified two porcine IL-12R subunits are indeed members of the superfamily of cytokine receptors.

### DOCUMENTS CITED

Chizzonite R, Truitt T, Desai B B, Nunes P, Podlaski F J, Stern A S, Gately M K. IL-12 receptor. I. Characterization of the receptor on phytohemagglutinin-activated human lymphoblasts. J. Immunol. May 15, 1992;148(10):3117-24.

Chua A O, Chizzonite R, Desai B B, Truitt T P, Nunes P, Minetti L J, Warrier R R, Presky D H, Levine J F, Gately M K, et al. Expression cloning of a human IL-12 receptor component. A new member of the cytokine receptor superfamily with strong homology to gp130. J. Immunol. Jul. 1, 1994; 153(1):128-36.

Desai, B. B., Quinn, P. M., Wolitzky, A. G., Mongini, P. K. A., Chizzonite, R., and Gately, M. K. 1992. IL-12 Receptor. II. Distribution and Regulation of Receptor Expression. J. Immunol. 148(10):3125-3132

Presky D H, Yang H, Minetti L J, Chua A O, Nabavi N, Wu C Y, Gately M K, Gubler U A functional interleukin 12 receptor complex is composed of two Beta-type cytokine receptor subunits. Proc. Natl. Acad. Sci. USA. Nov. 26, 1996;93(24):14002-7.

Residual type 1 immunity in patients genetically deficient for interleukin-12 receptor Beta-1 (IL-12RBeta-1): evidence for an IL-12RBeta-1-independent pathway of IL-12 responsiveness in human T cells. J. Exp. Med. Aug. 21, 2000;192(4):517-528

Verhagen C E, de Boer T, Smits H H, Verreck F A, Wierenga E A, Kurimoto M, Lammas D A, Kumararatne D S, Sanal O, Kroon F P, van Dissel J T, Sinigaglia F, Ottenhoff T H.

[From gene to disease; mutations in interleukin-12-receptor-beta 1- and interferon-gamma-receptor-1 lead to nontuberculous mycobacterial infections and salmonellosis]. Ned Tijdschr Geneeskd Sep. 16, 2000;144(38):1830-2.

[Article in Dutch] van Dissel J T, Ottenhoff T H. Afd. Infectieziekten, Leids Universitair Medisch Centrum, Leiden.

We claim:

1. A porcine IL-12 receptor encoded by the cDNA molecule subunits of the porcine beta-1 chain and porcine beta-2 chain.

2. A cDNA molecule encoding the porcine beta-1 chain of the porcine interleulkin-12 receptor, said cDNA molecule having a nucleotide sequence as shown in FIG. 1.

3. A cDNA molecule encoding the porcine beta-2 chain of the porcine interleukin-12 receptor, said cDNA sequence having a nucleotide sequence as shown in FIG. 5.

4. A method of identifying pigs capable of developing a strong cellular immune response to a given pathogen, said method comprising:
(a) specifying a given pathogen; and
(b) selecting pigs with allelic variants of an IL-12 receptor that confer on the pigs a superior ability compared to pigs without the variants, to develop a strong cellular response upon infection or vaccination against the pathogen.

5. A molecule with an amino acid sequence deduced from the cDNA sequence of claim 1, said amino acid sequence as shown in FIG. 1.

6. A molecule with an amino acid sequence deduced from the cDNA sequence of claim 2, said amino acid sequence as shown in FIG. 5.

7. A method for improving the cellular immune response of pig herds, said method comprising:
(a) selecting pigs with allelic variants of an IL-12 receptor that confer on the pigs a superior ability compared to pigs without the variants, to develop a strong cellular response upon infection or vaccination against the pathogen; and
(b) breeding the selected pigs according to methods known to those of skill in the art of pig breeding.

8. A molecule with an amino acid sequence deduced from the cDNA sequence of claim 3, said amino acid sequence having an allelic variant at position 1254 that results in a serine at amino acid position #342 as shown in FIG. 5.

9. The molecule of claim 8 wherein the variant result from a nucleotide change at position 1254 from A to C.

10. The molecule of claim 8 further characterized as having associate as a high IFN-g response or cell mediated immunity to an infectious agent in a pig.

11. The molecule of claim 8 further characterized as associated with a high IFN-g response or cell mediated immunity to the infectious agent PRRSV in a pig.

12. The molecule of claim 8 further characterized as associated with a high immune response to microorganims.

13. The molecule of claim 8 further characterized as associated with a high immune response to viruses.


SEQUENCE LISTING (12 sequences)

SEQ ID NO: 1 - DNA, 22 bp, Artificial Sequence
  Description of Artificial Sequence Primer
gactacacaagacaacagaatt

SEQ ID NO: 2 - DNA, 22 bp, Artificial Sequence
  Description of Artificial Sequence Primer
gactacaaaagacaacagattt

SEQ ID NO: 3 - DNA, 22 bp, Artificial Sequence
  Description of Artificial Sequence Primer
tattatgcagtgtggaattccc

SEQ ID NO: 4 - DNA, 22 bp, Artificial Sequence
  Description of Artificial Sequence Primer
gggaattccacactgcataata

SEQ ID NO: 5 - DNA, 22 bp, Artificial Sequence
  Description of Artificial Sequence Primer
gggaatgccacactgcagaata

SEQ ID NO: 6 - protein, 5 aa, Artificial Sequence
  Description of Artificial Sequence Synthetic peptide motif
WSXWS

SEQ ID NO: 7 - DNA, 3135 bp, Porcine sp.
  CDS (76)..(2271)
tgaggactggttggctgggggccttgctggtagcacagaagcttgtctggagctgctgatctctgcaaggaccggatgtggcaacgagtgaccaggctggtccccctcttcctcctcctgttcccgaggcagggtgctgaagcctgcagtaccagcaggtgctgtttccaggacccgccatatccggatgcagactcaggctcagcttctggccccagggacctaaactgctaccggatattcaacagagctggttatgaatgttcctgggagtatgagggccccacagctggggtcagccacttcctgagatgctgcctcaagtctgggcgctgttgctactttgccacaggctcagccaccacactgcagttttccgaccaggatggcgtagacgtgctcaatgctgtcactctctgggtggaatcccgggctgccaacaagacggagaagtcccccatggttaccttgaatctctacagctctgttaaatacgaccctcccccaggaaacatcaaggtatcaaggtcagcagggcagctgcacatggagtgggagaccccaacccgccaggatggtgctgagatacagttccgacaccggacacctggaagcccgtggatgctgggcgactgtggacgtcaggatgatgacgctggcttcgagtcatgcctatgccccctggagatggacatggcccaagaattccagctgcgacgacgacgacaccaggggccaggggtcccaggaggcccctggagcagctggagcagctctgtgtgcatcccccctgaaaaccccccacaggccaaggcgaatttctcagtggagcagctccgctcagatgggaggaggcaggtgaccttgaatgagcagctgccccagcttgagcttccggaaggctgccttgggaacaactctggagtggaaatgacctaccatgttcacctgcacatgctgtcctgcccttgtaaggccaaggccacaaggactctgcgcctaaggaaaaagctcgtcctctcaggcgctgcctatgacctggctctcatttcccggaatcgctttggccttggccccaaccagacatggcaccttcctgcctataaccattcaggaccagggactctgaatatcagtgttggagccaataggaccaccatgcattggccagtcctggcccagggcctgacgtactgcattgagtggcagccccagggccaggatgacagccttgtcaactgtaccttgatgacacccaaggaccaggaccctgctggaatggcagcctacagctggagccaagcacttggggcaatggggcagaaggtgtgttatcgcatcaccatctttgcctctgcacgtccagagaagcccacgtcatggtccacagtctcatcaacctactactttgcaggcaatgcctcaggagccgggagcccacagcatgtgtcggtgaagagactcaaccaggattccgtgtctgtggactggacaccatctctgctgagtgcctgccccggcgtcctgaaggagtacattgtgcgctgccatgatgaggacagcgaccaagtattcgagctgccagtgaagccaacagagacccgggtcacccttcaaggcctgcgggctggcgcagcctacatggtgcaggttcgagcagacacagccaagtggcagggctcctggagccagcccctgcgcttcagcatcgaagtccctgtttctgagttgcgcgatttgtccatcttcctggcatctttggggagcttcatgagcatccttctcctgggagtctttgggtaccttggcttgaacagggttgtaaggcatctgtgcccgcccctgcccacaccttgtgccagcactgccgttgagttctctgacagccaggggaagcaggtttggcagtggaccagcccagcagacttcccggaggaggtgtccccccaagaagccctggtggtgaacatgtcctgggacaaaggcgagggggttgacccagacacacctgggcctctcaaggagaagatggagctgcctctggatgcccctgagccggccctagacatggagctgcccttggagcacaggaggcaggcgcaaggacaccctgagcctgggactctgaggtctgggtggcacggtggtctggagggcagccctgcccagacagctggactcctgctgctcctgggagatctaaggcagactcccaggtttggttcccagggagaaaggggaacatctgcctcctcttacatagaggaagactgatgattgcaatcacgatggctggttattaagcacttactgcatacccagcctgtgccgcccagacactcatgttatgccctgaatccccataactgccttgaggtatgaggctgtcctatttacctatttctctgagtctcaccgaggctcagagagggtgagtgagtgccttgcctatggctacacagcaggtgcataagtcgaacctaggcggagtgactccgaccctggccactgcttccattcacacctactctgtcctgctgtcacacatcctgtggtgggaaccctagaaaccaccatgaaatggaagctgctaccccaagccagggcctggcacccatggggatagactagagggaggctcaaaggggttcatggtggtcctgggctgggcatcctgtaccagaagcactggatggggaatctgggtgcacagcgtcctcggccacatcgcagggtctccaggcttggaggatctaggtttagggaaaagaagtggggctcaaagcattctgagggaggtgatagctcctccctggcctcagtctccttctctgtaaaatgggcctgaggataatgttgtcctcacagagatgggggagatgtaaggcttaataaatttttcccctggagttctcattgtggctcagcagaaacgaatccaacaggaaccatgaggttgcgggttcgacccctggccttgctcagtgggttaaggatctggtgttgctgtgagcaagacacagctaggatcctacattgctgtggctgtggctgtggccagcagttgtagctccaattcgaccccaagcaagggaacctccacatgctgcaggtgtagccctaaaaaaaaaaaaa

SEQ ID NO: 8 - protein, 732 aa, Porcine sp.
MWQRVTRLVPLFLLLFPRQGAEACSTSRCCFQDPPYPDADSGSASGPRDLNCYRIFNRAGYECSWEYEGPTAGVSHFLRCCLKSGRCCYFATGSATTLQFSDQDGVDVLNAVTLWVESRAANKTEKSPMVTLNLYSSVKYDPPPGNIKVSRSAGQLHMEWETPTRQDGAEIQFRHRTPGSPWMLGDCGRQDDDAGFESCLCPLEMDMAQEFQLRRRRHQGPGVPGGPWSSWSSSVCIPPENPPQAKANFSVEQLRSDGRRQVTLNEQLPQLELPEGCLGNNSGVEMTYHVHLHMLSCPCKAKATRTLRLRKKLVLSGAAYDLALISRNRFGLGPNQTWHLPAYNHSGPGTLNISVGANRTTMHWPVLAQGLTYCIEWQPQGQDDSLVNCTLMTPKDQDPAGMAAYSWSQALGAMGQKVCYRITIFASARPEKPTSWSTVSSTYYFAGNASGAGSPQHVSVKRLNQDSVSVDWTPSLLSACPGVLKEYIVRCHDEDSDQVFELPVKPTETRVTLQGLRAGAAYMVQVRADTAKWQGSWSQPLRFSIEVPVSELRDLSIFLASLGSFMSILLLGVFGYLGLNRVVRHLCPPLPTPCASTAVEFSDSQGKQVWQWTSPADFPEEVSPQEALVVNMSWDKGEGVDPDTPGPLKEKMELPLDAPEPALDMELPLEHRRQAQGHPEPGTLRSGWHGGLEGSPAQTAGLLLLLGDLRQTPRFGSQGERGTSASSYIEED

SEQ ID NO: 9 - DNA, 2224 bp, Porcine sp.
  CDS (19)..(765)
gatctctgcaaggaccggatgtggcaacgagtgaccaggctggtccccctcttcctcctcctgttcccgaggcagggtgctgaagcctgcagtaccagcaggtgctgtttccaggacccgccatatccggatgcagactcaggctcagcttctggccccagggacctaaactgctaccggatattcaacagagctggttatgaatgttcctgggagtatgagggccccacagctggggtcagccacttcctgagatgctgcctcaagtctgggcgctgttgctactttgccacaggctcagccaccacactgcagttttccgaccaggatggcgtagacgtgctcaatgctgtcactctctgggtggaatcccgggctgccaacaagacggagaagtcccccatggttaccttgaatctctacagctctgttaaatacgaccctcccccaggaaacatcaaggtatcaaggtcagcagggcagctgcacatggagtgggagaccccaacccgccaggatggtgctgagatacagttccgacaccggacacctggaagcccgtggatgctgagtcatgcctatgccccctggagatggacatggcccaagaattccagctgcgacgacgacgacaccaggggccaggggtcccaggaggcccctggagcagctggagcagctctgtgtgcatcccccctgaaaaccccccacaggccaaggcgaatttctcagtggagcagctccgctcagatgggaggaggcaggtgaccttgaatgagcagctgccccagcttgagcttccggaaggctgccttgggaacaactctggagtggaaatgacctaccatgttcacctgcacatgctgtcctgcccttgtaaggccaaggccacaaggactctgcgcctaaggaaaaagctcgtcctctcaggcgctgcctatgacctggctctcatttcccggaatcgctttggccttggccccaaccagacatggcaccttcctgcctataaccattcaggaccagggactctgaatatcagtgttggagccaataggaccaccatgcattggccagtcctggcccagggcctgacgtactgcattgagtggcagccccagggccaggatgacagccttgtcaactgtaccttgatgacacccaaggaccaggaccctgctggaatggcagcctacagctggagccaagcacttggggcaatggggcagaaggtgtgttatcgcatcaccatctttgcctctgcacgtccagagaagcccacgtcatggtccacagtctcatcaacctactactttgcaggcaatgcctcaggagccgggagcccacagcatgtgtcggtgaagagactcaaccaggattccgtgtctgtggactggacaccatctctgctgagtgcctgccccggcgtcctgaaggagtacattgtgcgctgccatgatgaggacagcgaccaagtattcgagctgccagtgaagccaacagagacccgggtcacccttcaaggcctgcgggctggcgcagcctacatggtgcaggttcgagcagacacagccaagtggcagggctcctggagccagcccctgcgcttcagcatcgaagtccctgtttctgagttgcgcgatttgtccatcttcctggcatctttggggagcttcatgagcatccttctcctgggagtctttgggtaccttggcttgaacagggttgtaaggcatctgtgcccgcccctgcccacaccttgtgccagcactgccgttgagttctctgacagccaggggaagcaggtttggcagtggaccagcccagcagacttcccggaggaggtgtccccccaagaagccctggtggtgaacatgtcctgggacaaaggcgagggggttgacccagacacacctgggcctctcaaggagaagatggagctgcctctggatgcccctgagccggccctagacatggagctgcccttggagcacaggaggcaggcgcaaggacaccctgagcctgggactctgaggtctgggtggcacggtggtctggagggcagccctgcccagacagctggactcctgctgctcctgggagatctaaggcagactcccaggtttggttcccagggagaaaggggaacatctgcctcctcttacatagaggaagactgatgattgcaatcacgatggctggttattaagcacttactgcatac

SEQ ID NO: 10 - protein, 249 aa, Porcine sp.
MWQRVTRLVPLFLLLFPRQGAEACSTSRCCFQDPPYPDADSGSASGPRDLNCYRIFNRAGYECSWEYEGPTAGVSHFLRCCLKSGRCCYFATGSATTLQFSDQDGVDVLNAVTLWVESRAANKTEKSPMVTLNLYSSVKYDPPPGNIKVSRSAGQLHMEWETPTRQDGAEIQFRHRTPGSPWMLSHAYAPWRWTWPKNSSCDDDDTRGQGSQEAPGAAGAALCASPLKTPHRPRRISQWSSSAQMGGGR

SEQ ID NO: 11 - DNA, 4023 bp, Porcine sp.
  CDS (230)..(2812)
gaggggaagaggattctcgcagaggaaatgaaaagtgcacggtccccggagcagcaaagggcatgtcttgttcagggaaccgtctgaaggccagtgtgaatgacgcagagctgggaaggcaaaagattagagccagtgatggtggcaagacaggagatgtgaagccaggtagtggccgagagtccgatgtgggaaaatacagagaggtgtatcccagagttgactgttcatggcacgaacagtttgcgggtgctcatgggcccttatttttatcatcatgtcactcttggttaaggcaaaaatagatgtgtgcaagagaggggatgtgacggtgcagccttcccatgtaatttcacttggatcagctgtcaacatttcatgctctttgaagcccaggcaaggctgcttacaagtctccagtttaaacaagctaatcctctacaggttccacaggagaatccactttcagcgtggtcactccctcagttctcaagtcacaggtcttcccctgggtaccaccctgttcgtctgcaaactggcctgtagcagtaaggaggagattcgaatatgtggggccgagatctcggttggtgttgttccagaacagcctcaaaacgtatcttgtatgcagaagggagaacgtgggacagtggcctgcagctgggacagaggacgagatacccatctatatactgcatatactttacaattaaatggaccaaaaaatttaacttggcagaagcaatgtagcgattattattgtgactctttggaccttggaatcaacctaccccctgaatcacctgaatctagttacacagcccaggttactgccatcaatagtctagggactgcgtcttcatttccatgcacattcacattgttggatgtagtgaggcctcttcctccgtgggacatcagaatcaaatgtgtaaatgcctccgtgagcacatgtaccctgcaatggagagatgaggggctagtgctgcttaatcgactcagataccggcctgtttacagcagatcctggaatatggttaatgctacaaatgccaaaggaagacatgatttggtggatctgaaaccctttacagaatatgaatttcagatttcctctaagccacatcttcagaagggtagatggagtgattggagtgaatcattgagaactcaaactccagagaaagagcctacagggatgttagacgtctggtacatgaaacagcacattgactacaaaagacaacagatttatcttttctggaagaatctgagtctatcagaggcaagaggaaaaatcctccactatcaagtgaccttgcaggaggtcgcagaggggaatgccacactgcagaatatcactgaacgcaactcctggacctggaccatacccagaactggcatctgggctgcggccgtgtctgcagctaactccaaaggcagttccctgcccactcgtattaacatagcagacctgtgcggagcagggttgctggctcctcagcaggtttctgcaaacccagagggctcggacaacctcctggtgaagtggacgtctccgggggaaggtgccactgctgtgcaggagtatgtggtggagtggagggagctccatctgaggggtggcatgcagccccctctaagctggctgcggagtcccccctacaacacgtccaccctcatctcagacaacataaaaccctacatctgttatgaaatccgagtgcacgcactttcaggggaccagggaggatgcagctccatccggggtgacttgaagcataaagcaccactgagtggcccccacattaatgccatctcagaggaaaaggggagcattttaatttcatgggacgaaattccagcccaggagcaaatgggctgcatcctccattacaggatctattggaaggaacgggactccgattcccagcctcagctctgtgaaattccctatagagtctcccccaaatcacatcccatcaatagcctgcagcccagagtgacatacgtcctgtggatgacagctctgacagctgctggcgaaagcccccaaggaaatgagagggaattttgtctgcaaggtaaagccaattggagcacatttgtagcaccaagcatttgcattgctgtcatcacagtgggcgttttctcaatgcgttgcttccggcaaaaggtatttgttctccttttggccctcagacctcagtggtgtagcagagaaattccagacccagccaatagcacttgggccaagaaatatcccattgtggaggagaagaaacagctgtccctggataggctcctggcagactggcccactcctgaagaacctgagcccctggtcatcaatgaagtccttcctcaagtgactccagtcttcagacgcccccatcatcccaactggccaggaaagggacaaaggctccaaggtcaccacgcctctgaggaagacacagggtccagtgcctcaagtccaccacctccaagggctctcacagcagagacaggacccgcagtggatctgtacaaggtgctggggagccgacgccctgactcaaagccgggaaacccagtcagccacttgacggtcctccccgtggactacctgcctacccatgagggttacttaccctccaacatggattatctcccttcacatgaagctcccataacagactctctggaagaactgcctcagcacatctctctttccgttttcccctcaaattcccttcacccgctgaccttctcctgtggtgagaagttgactctggatcagttgaagatggggtgtggctctctcatgctctgagtggtgaagcgtgatgtttggaaagtcagtgtggccccagccagcaaagcatggcctgtctacccagcctcctgctccgggatcatcgcccctgggcgctaccattaggtctggttccaactagagggcaggcaaaccagcttagggcaaatctcaggaactgagagttggctgtaacccagatacctcacactgccttctcctgagcctcgagactacgtcccccactctgtggacctggagactccaacttgggtacctgcaactctcttggctatgttggccagaaagggacaaggcaagagtagaaaccaaaactcgttgaatcagagctgcagctaccagcctacaccagccacagcaacacaggatccgagccacctgtgccacctacaccatagctcacggcaacatcggatccttaacccactgagtgaggccaggaattgaacctgcatcctcacagacaccatgtcaggttcttaacccactgagccacaacgggaactccacattttttttttttttagggctgcacctgtggcaatatggaagtttccagcctaggggtcaaattggagctacagctaccagcctgtgccacagctacagcaatgccagatctgagccacaagtgcgacctacaccacagctcgtggcaacaccagatccttaacccactgagcaaggccaaggattgaacccacatcctcatggacactagtcggatttgcttccactaagccaccaagggaactcccagatttttaaaaataataaattctacagtactacatgatccattgttggatgaacccatggatacagaagcacagatacagaaggccaactaaaagttatacgctgattttcaactgtacggggagctggtgctcctaaccccatgttgttcaagggtcatcgtataaagcagctactacaaaccaggtcttatgtctagatgagaactgtccaccacatcggccattgaccactttggaatatacctctagtatgactgaggagctggtttttattgtaattaaaatagtcacatatggctcttggctactatactggagagcacaactctaaatgactattaagacaatatgaagaaaatttcatttttgtctaattgcaattaaaagaagatgttaaaatatccaaatgaaaataaaattaatactttcacaaaaaaaaaaaaa

SEQ ID NO: 12 - protein, 861 aa, Porcine sp.
MARTVCGCSWALIFIIMSLLVKAKIDVCKRGDVTVQPSHVISLGSAVNISCSLKPRQGCLQVSSLNKLILYRFHRRIHFQRGHSLSSQVTGLPLGTTLFVCKLACSSKEEIRICGAEISVGVVPEQPQNVSCMQKGERGTVACSWDRGRDTHLYTAYTLQLNGPKNLTWQKQCSDYYCDSLDLGINLPPESPESSYTAQVTAINSLGTASSFPCTFTLLDVVRPLPPWDIRIKCVNASVSTCTLQWRDEGLVLLNRLRYRPVYSRSWNMVNATNAKGRHDLVDLKPFTEYEFQISSKPHLQKGRWSDWSESLRTQTPEKEPTGMLDVWYMKQHIDYKRQQIYLFWKNLSLSEARGKILHYQVTLQEVAEGNATLQNITERNSWTWTIPRTGIWAAAVSAANSKGSSLPTRINIADLCGAGLLAPQQVSANPEGSDNLLVKWTSPGEGATAVQEYVVEWRELHLRGGMQPPLSWLRSPPYNTSTLISDNIKPYICYEIRVHALSGDQGGCSSIRGDLKHKAPLSGPHINAISEEKGSILISWDEIPAQEQMGCILHYRIYWKERDSDSQPQLCEIPYRVSPKSHPINSLQPRVTYVLWMTALTAAGESPQGNEREFCLQGKANWSTFVAPSICIAVITVGVFSMRCFRQKVFVLLLALRPQWCSREIPDPANSTWAKKYPIVEEKKQLSLDRLLADWPTPEEPEPLVINEVLPQVTPVFRRPHHPNWPGKGQRLQGHHASEEDTGSSASSPPPPRALTAETGPAVDLYKVLGSRRPDSKPGNPVSHLTVLPVDYLPTHEGYLPSNMDYLPSHEAPITDSLEELPQHISLSVFPSNSLHPLTFSCGEKLTLDQLKMGCGSLML